(19) United States
(12) Patent Application Publication
(10) Pub. No.: US 2004/0122049 A1
(43) Pub. Date: Jun. 24, 2004

(54) Novel piperidine derivatives as modulators of chemokine receptor

(76) Inventors: Jeremy Burrows, Macclesfield (GB); John Cumming, Macclesfield (GB)

Correspondence Address: Fish & Richardson, 225 Franklin Street, Boston, MA, 02110-2804, US

(21) Appl. No.: 10/473,258

(22) PCT Filed: Mar. 26, 2002
(86) PCT No.: PCT/SE02/00598

(30) Foreign Application Priority Data
Mar. 30, 2001 (GB) 0108046.4

(51) Int. Cl. C 07D 4 1/02; A61K 31/454; A61K 31/445; C07D 211/56

(52) U.S. Cl. 514/317; 514/326; 546/207; 546/223

(57) ABSTRACT

The invention provides a compound of formula (I) wherein: R¹ is a group selected from: (a), (b) and (c) or a pharmaceutically acceptable salt thereof or a solvate thereof; compositions containing these compounds, processes for preparing them and their use as modulators of chemokine activity (especially CCR5 activity).

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in EP-A1-1013276, WO00/08013, WO99/38514 and WO99/04794. Piperidine oxime derivatives are disclosed in GB 1538542.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and IO (MIP-1α and MIP-1β).

Studies have demonstrated that the actions of the chemokines are mediated by subfamilies-of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1a and MIP-1b and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I):

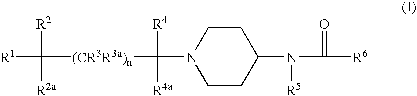

wherein:

R¹ is a group selected from:

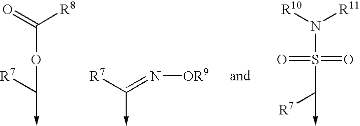

R², R^{2a}, R⁴ and R^{4a} are, independently, hydrogen or C₁₋₄ alkyl;

R³ and R^{3a} are, independently, hydrogen, C₁₋₄ alkyl or C₁₋₄ alkoxy;

n is 0 or 1;

R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;

R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;

R⁷ is phenyl, heteroaryl, phenyl(C₁₋₄ alkyl) or heteroaryl(C₁₋₄ alkyl);

R⁸ is C₁₋₈ alkyl, OR¹², NR¹³R¹⁴, phenyl, heteroaryl, phenyl(C₁₋₂)alkyl or heteroaryl(C₁₋₂)alkyl;

R⁹, R¹⁰ and R¹¹ are, independently, hydrogen, C₁₋₆ alkyl (optionally substituted by C₁₋₆ alkoxy, phenyl or heteroaryl), phenyl or heteroaryl; or R¹⁰ and R¹¹ may join to form a 5- or 6-membered ring which may additionally include an oxygen atom or a further nitrogen atom, said ring being optionally substituted with C₁₋₄ alkyl, C(O)H or C(O)(C₁₋₄ alkyl);

R¹² and R¹³ are C₁₋₈ alkyl (optionally substituted by halogen, OH, cyano, C₁₋₆ alkoxy, C₁₋₆ hydroxyalkoxy, C₁₋₆ alkylthio, C₃₋₆ cycloalkyl, NR¹⁵R¹⁶, C(O)NH(OH), NHC(O)(C₁₋₄ alkyl), heterocyclyl, phenyl or heteroaryl), C₃₋₆ alkenyl, C₃₋₆ alkynyl, C₃₋₆ cycloalkyl (optionally substituted by C₁₋₆ alkyl), phenyl, heteroaryl or heterocyclyl;

R¹⁴ is hydrogen or is independently selected from the list of options recited for R¹³;

or R¹³ and R¹⁴ join to form a 5, 6, 7 or 8-membered monocyclic or bicyclic ring system which is optionally unsaturated, optionally includes a further nitrogen atom or also includes an oxygen or sulphur atom, and which is optionally substituted by OH, C₁₋₆ alkyl or C₁₋₆ hydroxyalkyl; %

R¹⁵ and R¹⁶ are, independently, hydrogen or C₁₋₆ alkyl;

wherein the phenyl, heteroaryl and heterocyclyl rings of any of the foregoing are independently optionally substituted by halo, cyano, nitro, oxo, hydroxy, C₁₋₄ alkyl, C₁₋₄ hydroxyalkyl, C₁₋₄ alkoxy, S(O)ₘC₁₋₄ alkyl, S(O)₂NR¹⁷ R¹⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;

R¹⁷ and R¹⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);

m, p and q are, independently, 0, 1 or 2;

or a pharmaceutically acceptable salt thereof or a solvate thereof;

provided that when R¹ is

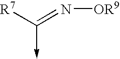

n is 0 or 1; R², R^{2a}, R³, R^{3a}, R⁴, R^{4a}, R⁵ and R⁹ are all hydrogen; and R⁶ is unsubstituted phenyl; then R⁷ is not optionally substituted phenyl, or a salt thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate or p-toluenesulphonate.

The compounds of the invention may exist as solvates (such as hydrates) and the present invention covers all such solvates.

Alkyl groups and moieties are straight or branched chain and are, for example, methyl, ethyl, n-propyl or iso-propyl.

Alkenyl and alkynyl groups and moieties are, for example, vinyl, allyl or propargyl.

Cycloalkyl is a mono-, bi- or tri-cyclic structure such as, for example, cyclopropyl, cyclopentyl, cyclohexyl or adamantyl.

Cycloalkenyl comprises one double bond and is, for example, cyclopentenyl or cyclohexenyl.

Acyl is, for example, carbonyl substituted by C₁₋₆ alkyl or optionally substituted phenyl.

Heterocyclyl is a non-aromatic 3, 4, 5 or 6 membered ring comprising at least one heteroatom selected from the group comprising nitrogen, oxygen and sulphur. Heterocyclyl is, for example, aziridinyl, azetidinyl, oxetanyl, piperidinyl, 4,5-dihydro-oxazolyl, 4,5-dihydroimidazolyl, morpholinyl, thiomorpholinyl, pyrrolidinyl, piperazinyl or tetrahydrofuryl.

Heteroaryl is an aromatic 5 or 6 membered ring comprising at least one heteroatom selected from the group comprising nitrogen, oxygen and sulphur. Heteroaryl is, for example, pyrrolyl, imidazolyl, pyrazolyl, 1,2,3-triazolyl, 1,2,4-triazolyl, oxazolyl, isoxazolyl, oxadiazolyl, thiazolyl, isothiazolyl, thiadiazolyl, pyridinyl, pyrimidinyl, pyrazinyl, pyridazinyl, thienyl, furyl, quinolinyl, isoquinolinyl, dihydroisoquinolinyl, quinazolinyl, quinoxalinyl, indolyl, isoindolyl, benzimidazolyl, benzo[b]furyl, benzo[b]thienyl, phthalazinyl, indanyl, benzthiazolyl or cinnolinyl.

Phenylalkyl is, for example, benzyl, 1-(phenyl)ethyl or 2-(phenyl)ethyl.

Heteroarylalkyl is, for example, pyridinylmethyl, pyrimidinylmethyl or 2-(pyridinyl)ethyl.

The group S(O)₂NR¹⁷R¹⁸ is, for example, S(O)₂NH₂, S(O)₂NH(C₁₋₄ alkyl), S(O)₂N(C₁₋₄ alkyl)₂, S(O)₂(4-C(O)H-piperazin-1-yl) or S(O)₂(4-C(O)CH₃-piperazin-1-yl).

Phenyl(C₁₋₂ alkyl)NH is, for example, benzylamino. Heteroaryl(C₁₋₂ alkyl)NH is, for example, pyridinylCH₂NH, pyrimidinylCH₂NH or pyridinylCH(CH₃)NH.

In one aspect R¹ is a group selected from:

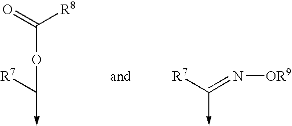

In a further aspect R¹ is CHR⁷OC(O)NR¹³R¹⁴ wherein R¹³ and R¹⁴ are as defined above. In yet another aspect R¹⁴ is not hydrogen. In a further aspect R¹³ and R¹⁴ join to form a ring system as defined above.

In a still further aspect n is 0.

In yet another aspect R⁴ and R^{4a} are hydrogen or methyl; for example R⁴ is hydrogen and R^{4a} is hydrogen or methyl.

In a further aspect R⁴ is hydrogen, R^{4a} is hydrogen or methyl, and R³ and R^{3a} are both hydrogen.

In a still further aspect n is 0 and R², R^{2a}, R⁴ and R^{4a} are all hydrogen; R^{4a} can also be methyl.

In another aspect n is 1 and R², R^{2a}, R³, R^{3a}, R⁴ and R^{4a} are all hydrogen; R^{4a} can also be methyl.

In yet another aspect R⁵ is hydrogen or C₁₋₄ alkyl (such as methyl, ethyl or iso-propyl), C₃₋₄ alkenyl (for example allyl), C₃₋₄ alkynyl (for example propargyl), C₃₋₇ cycloalkyl (for example cyclopropyl) or C₃₋₇ cycloalkyl(C₁₋₄ alkyl) (for example cyclopropylCH2). The variable R⁵ can be methyl, ethyl or allyl. It is preferred that R⁵ is ethyl.

In a further aspect R⁶ is preferably optionally substituted benzyl, especially benzyl singly substituted (such as in the 4-position) by S(O)₂(C₁₋₄)alkyl (such as S(O)₂CH₃) or S(O)₂NR⁹R¹⁰ {R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl)} (such as S(O)₂NH₂, S(O)₂NH(CH₃), S(O)₂N(CH₃)₂, S(O)₂(4-C(O)H-piperazin-1-yl) or S(O)₂(4-C(O)CH₃-piperazin-1-yl).

In a still further aspect R⁶ is preferably optionally substituted benzyl, especially benzyl singly substituted (such as in the 4-position) by halo (such as fluoro) or S(O)₂(C₁₋₄)alkyl (such as S(O)₂CH₃).

In another aspect R⁷ is optionally substituted phenyl (especially optionally substituted by halo, cyano, methyl, ethyl, methoxy, ethoxy, NH₂, NHCH₃, N(CH₃)₂, CF₃, CHF₂, CH₂F, CH₂CF₃ or OCF₃). In another aspect R⁷ is optionally substituted phenyl (especially optionally substituted by halogen or CF₃). For example R⁷ is unsubstituted phenyl, 3-fluorophenyl, 3-chlorophenyl, 4-fluorophenyl or 4-CF₃-phenyl.

In yet another aspect R⁸ is C₁₋₆ alkyl, C₁₋₆ alkoxy, NR¹³R¹⁴, C₃₋₇ cycloalkyl (optionally substituted by C₁₋₄ alkyl) or heteroaryl; R¹³ is C₁₋₈ alkyl (optionally substituted by halogen, cyano, hydroxy, NH₂, N(C₁₋₄ alkyl)₂, C₁₋₄ alkoxy, C₁₋₄ thioalkyl, C₃₋₇ cycloalkyl, heterocyclyl, phenyl, heteroaryl, NHC(O)(C₁₋₄ alkyl) or C(O)NHOH), C₃₋₆ alkenyl, C₃₋₆ alkynyl, phenyl or heteroaryl; R¹⁴ is hydrogen, C₁₋₈ alkyl (optionally substituted by cyano or hydroxy) or C₃₋₆ alkenyl; or R¹³ and R¹⁴ together with the nitrogen to which hey are attached form a oxiranyl, pyrrolidinyl, piperidinyl, morpholinyl, dihydropyrrolyl, tetrahydropyridinyl, piperazinyl, thiomorpholinyl, homopiperazinyl or homopiperidinyl ring all of which are optionally substituted by hydroxy, C₁₋₄ alkyl or C₁₋₄ hydroxyalkyl; wherein phenyl is optionally substituted by halogen, cyano, hydroxy or C₁₋₆ alkyl; and heteroaryl is optionally substituted by oxo, halogen, cyano, hydroxy or C₁₋₆ alkyl.

In a further aspect R⁸ is C₁₋₆ alkyl, C₁₋₆ alkoxy, NR¹³R¹⁴, C₃₋₇ cycloalkyl (optionally substituted by C₁₋₄ alkyl) or heteroaryl; R¹³ is C₁₋₈ alkyl (optionally substituted by halogen, cyano, hydroxy, NH₂, N(C₁₋₄ alkyl)₂, C₁₋₄ alkoxy, C₁₋₄ thioalkyl, C₃₋₇ cycloalkyl, heterocyclyl, phenyl, heteroaryl, NHC(O)(C₁₋₄ alkyl) or C(O)NHOH), C₃₋₆ alkenyl, C₃₋₆ alkynyl, phenyl or heteroaryl; and R¹⁴ is hydrogen, C₁₋₈ alkyl (optionally substituted by cyano or hydroxy) or C₃₋₆ alkenyl.

In a still further aspect R⁹ is C₁₋₄ alkyl (such as ethyl) or C₃₋₄ alkenyl (such as allyl).

In yet another aspect R¹⁰ and R¹¹ are, independently, hydrogen or C₁₋₄ alkyl (such as methyl).

In one aspect the present invention provides a compound of formula (Ia):

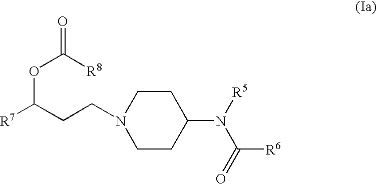

wherein R⁵, R⁶, R⁷ and R⁸ are as hereinbefore defined.

In one aspect the present invention provides a compound of formula (Ib):

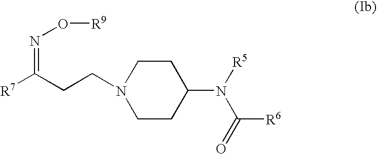

wherein R⁵, R⁶, R⁷ and R⁹ are as hereinbefore defined, provided that when R⁵ and R⁹ are both hydrogen; and R⁶ is unsubstituted phenyl; then R⁷ is not optionally substituted phenyl, or a salt thereof.

The following compounds illustrate the invention.

TABLE I
All compounds in Table I are of formula (Ia) below.

| | |
|---|---|
| | (Ia) |
| 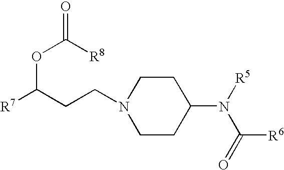 | |

| Compound | | | | | LCMS |
|---|---|---|---|---|---|
| No. | R⁸ | R⁷ | R⁵ | R⁶ | (MH+) |
| 1 | pyrrolidin-1-yl | Ph-4-F | Et | CH₂Ph-4-F | 514 |
| 2 | OCH₃ | Ph-4-F | Et | CH₂Ph-4-SO₂Me | 535 |
| 3 | N(CH₂CH₃)₂ | Ph-4-F | Et | CH₂Ph-4-F | 516 |
| 4 | NH(CH₂)₂(pyrrolidin-1-yl) | Ph | Et | CH₂Ph-4-SO₂Me | 599 |
| 5 | piperidin-1-yl | Ph | Et | CH₂Ph-4-SO₂Me | 570 |
| 6 | NHCH₂CF₃ | Ph | Et | CH₂Ph-4-SO₂Me | 584 |
| 7 | pyrrolidin-1-yl | Ph | Et | CH₂Ph-4-SO₂Me | 556 |
| 8 | morpholin-4-yl | Ph | Et | CH₂Ph-4-SO₂Me | 572 |
| 9 | 4-CH₃-piperazin-1-yl | Ph | Et | CH₂Ph-4-SO₂Me | 585 |
| 10 | NHCH₂Ph | Ph | Et | CH₂Ph-4-SO₂Me | 592 |
| 11 | NHCH₂Ph-4-F | Ph | Et | CH₂Ph-4-SO₂Me | 610 |
| 12 | NH(CH₂)₂OMe | Ph | Et | CH₂Ph-4-SO₂Me | 560 |
| 13 | N(Me)(CH₂)₂OMe | Ph | Et | CH₂Ph-4-SO₂Me | 574 |
| 14 | NHCH₂(pyridin-2-yl) | Ph | Et | CH₂Ph-4-SO₂Me | 593 |
| 15 | NHCH₂(pyridin-3-yl) | Ph | Et | CH₂Ph-4-SO₂Me | 593 |
| 16 | NH(cyclopropyl) | Ph | Et | CH₂Ph-4-SO₂Me | |
| 17 | NH(cyclobutyl) | Ph | Et | CH₂Ph-4-SO₂Me | |
| 18 | NH(cyclopentyl) | Ph | Et | CH₂Ph-4-SO₂Me | |
| 19 | NH(cyclohexyl) | Ph | Et | CH₂Ph-4-SO₂Me | |
| 20 | N(CH₂CN)₂ | Ph | Et | CH₂Ph-4-SO₂Me | 580 |
| 21 | N(CH₃)((CH₂)₂CN) | Ph | Et | CH₂Ph-4-SO₂Me | 569 |
| 22 | N(CH₃)((CH₂)₂OH) | Ph | Et | CH₂Ph-4-SO₂Me | 560 |
| 23 | N(CH₂CH₃)((CH₂)₂OH) | Ph | Et | CH₂Ph-4-SO₂Me | 574 |
| 24 | N(CH₂OH)₂ | Ph | Et | CH₂Ph-4-SO₂Me | 590 |
| 25 | NH(1,3,4-thiadiazol-2-yl) | Ph | Et | CH₂Ph-4-SO₂Me | |
| 26 | NH(3-CH₃-isoxazol-5-yl) | Ph | Et | CH₂Ph-4-SO₂Me | |
| 27 | NH(5-CH₃-isoxazol-3-yl) | Ph | Et | CH₂Ph-4-SO₂Me | |
| 28 | NH(fur-2-yl-CH₂) | Ph | Et | CH₂Ph-4-SO₂Me | 582 |
| 29 | NH(CCH₃(CH₂OH)₂) | Ph | Et | CH₂Ph-4-SO₂Me | 590 |
| 30 | oxiran-1-yl | Ph | Et | CH₂Ph-4-SO₂Me | |
| 31 | 2,5-dihydropyrrol-1-yl | Ph | Et | CH₂Ph-4-SO₂Me | |
| 32 | NH(1,2,4-1H-triazol-3-yl) | Ph | Et | CH₂Ph-4-SO₂Me | |
| 33 | NH(1H-pyrazol-3-yl) | Ph | Et | CH₂Ph-4-SO₂Me | 568 |
| 34 | NH(1H-4-CN-pyrazol-3-yl) | Ph | Et | CH₂Ph-4-SO₂Me | |
| 35 | NH(tetrahydrofuran-2-ylCH₂) | Ph | Et | CH₂Ph-4-SO₂Me | |
| 36 | 1,2,3,6-tetrahydropyridin-1-yl | Ph | Et | CH₂Ph-4-SO₂Me | |
| 37 | piperazin-1-yl | Ph | Et | CH₂Ph-4-SO₂Me | 571 |
| 38 | thiomorpholin-4-yl | Ph | Et | CH₂Ph-4-SO₂Me | 588 |
| 39 | 4-OH-piperidin-1-yl | Ph | Et | CH₂Ph-4-SO₂Me | 586 |
| 40 | 4-CH₃-piperidin-1-yl | Ph | Et | CH₂Ph-4-SO₂Me | 584 |
| 41 | NH(pyrimidin-2-yl) | Ph | Et | CH₂Ph-4-SO₂Me | 580 |
| 42 | NH(4-CH₃-pyrimidin-2-yl) | Ph | Et | CH₂Ph-4-SO₂Me | |
| 43 | NH(pyrimidin-4-yl) | Ph | Et | CH₂Ph-4-SO₂Me | 580 |
| 44 | NH(pyridazin-2-yl) | Ph | Et | CH₂Ph-4-SO₂Me | |
| 45 | NCH₃(pyridin-2-yl) | Ph | Et | CH₂Ph-4-SO₂Me | |
| 46 | NH(pyridin-2-yl) | Ph | Et | CH₂Ph-4-SO₂Me | 579 |
| 47 | NH(3-OH-pyridin-2-yl) | Ph | Et | CH₂Ph-4-SO₂Me | 595 |
| 48 | NH(3-CH₃-pyridin-2-yl) | Ph | Et | CH₂Ph-4-SO₂Me | 593 |
| 49 | NH(4-CH₃-pyridin-2-yl) | Ph | Et | CH₂Ph-4-SO₂Me | |
| 50 | NH(5-CH₃-pyridin-2-yl) | Ph | Et | CH₂Ph-4-SO₂Me | |
| 51 | NH(pyridin-2-yl-CH₂) | Ph | Et | CH₂Ph-4-SO₂Me | 593 |
| 52 | NH(pyridin-3-yl) | Ph | Et | CH₂Ph-4-SO₂Me | 579 |
| 53 | NH(pyridin-3-yl-CH₂) | Ph | Et | CH₂Ph-4-SO₂Me | 593 |
| 54 | NH(pyridin-4-yl) | Ph | Et | CH₂Ph-4-SO₂Me | |
| 55 | NH(pyridin-4-yl-CH₂) | Ph | Et | CH₂Ph-4-SO₂Me | 593 |
| 56 | NH(1,2,4-triazin-3-yl) | Ph | Et | CH₂Ph-4-SO₂Me | 581 |
| 57 | homopiperazin-1-yl | Ph | Et | CH₂Ph-4-SO₂Me | 585 |
| 58 | homopiperidin-1-yl | Ph | Et | CH₂Ph-4-SO₂Me | 583 |
| 59 | NH-phenyl | Ph | Et | CH₂Ph-4-SO₂Me | 578 |
| 60 | NH(2-OH—C₆H₄) | Ph | Et | CH₂Ph-4-SO₂Me | |
| 61 | NH(2-CH₃—C₆H₄) | Ph | Et | CH₂Ph-4-SO₂Me | 592 |
| 62 | NH(3-OH—C₆H₄) | Ph | Et | CH₂Ph-4-SO₂Me | |
| 63 | NH(3-CH₃—C₆H₄) | Ph | Et | CH₂Ph-4-SO₂Me | 592 |
| 64 | NH(4-OH—C₆H₄) | Ph | Et | CH₂Ph-4-SO₂Me | 594 |
| 65 | NH(3-CH₃—C₆H₄) | Ph | Et | CH₂Ph-4-SO₂Me | 592 |
| 66 | NHC(CH₃)₂CH₂OH | Ph | Et | CH₂Ph-4-SO₂Me | 574 |
| 67 | NHCH₂C(CH₃)₂NH₂ | Ph | Et | CH₂Ph-4-SO₂Me | 573 |
| 68 | NHC(CH₃)₂CH₂CH₃ | Ph | Et | CH₂Ph-4-SO₂Me | 572 |
| 69 | NHCH(CH₃)CH(CH₃)₂ | Ph | Et | CH₂Ph-4-SO₂Me | |
| 70 | NHCH(CH₃)₂ | Ph | Et | CH₂Ph-4-SO₂Me | |
| 71 | NHCH(CH₃)CH₂OCH₃ | Ph | Et | CH₂Ph-4-SO₂Me | 574 |
| 72 | NHCH(CH₃)CH₂OH | Ph | Et | CH₂Ph-4-SO₂Me | 560 |
| 73 | NHCH(CH₃)CH₂CH(CH₃)₂ | Ph | Et | CH₂Ph-4-SO₂Me | 586 |
| 74 | NHCH(CH₃)CH₂CH₃ | Ph | Et | CH₂Ph-4-SO₂Me | |
| 75 | NHCH(CH₂OH)CH₂CH₃ | Ph | Et | CH₂Ph-4-SO₂Me | 574 |
| 76 | NHCH(CH₂CH₃)₂ | Ph | Et | CH₂Ph-4-SO₂Me | |
| 77 | NHCH₃ | Ph | Et | CH₂Ph-4-SO₂Me | 516 |
| 78 | NHCH₂CF₃ | Ph | Et | CH₂Ph-4-SO₂Me | |
| 79 | NHCH₂C(CH₃)₃ | Ph | Et | CH₂Ph-4-SO₂Me | 572 |
| 80 | NHCH₂CH(OCH₃)₂ | Ph | Et | CH₂Ph-4-SO₂Me | |
| 81 | NHCH₂CH(OH)CH₃ | Ph | Et | CH₂Ph-4-SO₂Me | 560 |
| 82 | NHCH₂CH(OH)CH₂OH | Ph | Et | CH₂Ph-4-SO₂Me | 576 |
| 83 | NHCH₂CH(OH)CH₂NH₂ | Ph | Et | CH₂Ph-4-SO₂Me | 575 |
| 84 | NHCH₂CH(CH₃)₂ | Ph | Et | CH₂Ph-4-SO₂Me | |
| 85 | NHCH₂CH₃ | Ph | Et | CH₂Ph-4-SO₂Me | |
| 86 | NH(CH₂)₂NHC(O)CH₃ | Ph | Et | CH₂Ph-4-SO₂Me | 587 |
| 87 | N(CH₃)(CH₂)₂NH₂ | Ph | Et | CH₂Ph-4-SO₂Me | 559 |
| 88 | N(CH₂CH₃)(CH₂)₂NH₂ | Ph | Et | CH₂Ph-4-SO₂Me | 573 |
| 89 | N((CH₂)₂OH)(CH₂)₂NH₂ | Ph | Et | CH₂Ph-4-SO₂Me | 589 |
| 90 | N((CH₂)₂CH₃)(CH₂)₂NH₂ | Ph | Et | CH₂Ph-4-SO₂Me | |
| 91 | NH(CH₂)₂N(CH₃)₂ | Ph | Et | CH₂Ph-4-SO₂Me | 573 |
| 92 | NH(CH₂)₂OCH₃ | Ph | Et | CH₂Ph-4-SO₂Me | 560 |
| 93 | NH(CH₂)₂O(CH₂)₂OH | Ph | Et | CH₂Ph-4-SO₂Me | 590 |
| 94 | NH(CH₂)₂OH | Ph | Et | CH₂Ph-4-SO₂Me | 546 |
| 95 | NHCH₂C≡CH | Ph | Et | CH₂Ph-4-SO₂Me | |
| 96 | NH(CH₂)₂C(CH₃)₃ | Ph | Et | CH₂Ph-4-SO₂Me | 586 |
| 97 | NH(CH₂)₂CH(CH₃)₂ | Ph | Et | CH₂Ph-4-SO₂Me | 572 |
| 98 | NH(CH₂)₃N(CH₃)₂ | Ph | Et | CH₂Ph-4-SO₂Me | 587 |
| 99 | NH(CH₂)₃OCH₂CH₃ | Ph | Et | CH₂Ph-4-SO₂Me | |
| 100 | NH(CH₂)₃OH | Ph | Et | CH₂Ph-4-SO₂Me | 560 |
| 101 | NH(CH₂)₄OH | Ph | Et | CH₂Ph-4-SO₂Me | 574 |
| 102 | NH(CH₂)₄CH₃ | Ph | Et | CH₂Ph-4-SO₂Me | 572 |
| 103 | NH(CH₂)₅OH | Ph | Et | CH₂Ph-4-SO₂Me | 588 |
| 104 | NH(CH₂)₅CH₃ | Ph | Et | CH₂Ph-4-SO₂Me | |
| 105 | N(CH₃)phenyl | Ph | Et | CH₂Ph-4-SO₂Me | |
| 106 | N(CH₃)₂ | Ph | Et | CH₂Ph-4-SO₂Me | |
| 107 | N(CH₃)CH₂C≡CH | Ph | Et | CH₂Ph-4-SO₂Me | |
| 108 | N(CH₃)CH₂CH═CH₂ | Ph | Et | CH₂Ph-4-SO₂Me | |
| 109 | N(CH₂CH═CH₂)₂ | Ph | Et | CH₂Ph-4-SO₂Me | 582 |
| 110 | N(CH(CH₃)₂)₂ | Ph | Et | CH₂Ph-4-SO₂Me | |
| 111 | N(CH₃)CH₂CH₃ | Ph | Et | CH₂Ph-4-SO₂Me | |
| 112 | N(CH₃)(CH₂)₂CH₃ | Ph | Et | CH₂Ph-4-SO₂Me | 558 |
| 113 | N((CH₂)₂CH₃)₂ | Ph | Et | CH₂Ph-4-SO₂Me | 586 |
| 114 | N(CH₃)(CH₂)₃CH₃ | Ph | Et | CH₂Ph-4-SO₂Me | 572 |
| 115 | N(CH₂CH₃)(CH₂)₃CH₃ | Ph | Et | CH₂Ph-4-SO₂Me | 586 |
| 116 | NH(CH₂)₃CH₃ | Ph | Et | CH₂Ph-4-SO₂Me | 580 |
| 117 | 3-OH-piperidin-1-yl | Ph | Et | CH₂Ph-4-SO₂Me | 586 |
| 118 | NH(CH₂)₂CN | Ph | Et | CH₂Ph-4-SO₂Me | |
| 119 | NH(CH₂)₂SCH₂CH₃ | Ph | Et | CH₂Ph-4-SO₂Me | |
| 120 | NH(CH₂)₃OCH₃ | Ph | Et | CH₂Ph-4-SO₂Me | 574 |
| 121 | N(CH₃)CH₂CH(CH₃)₂ | Ph | Et | CH₂Ph-4-SO₂Me | 572 |
| 122 | N(CH₂CH₃)CH(CH₃)₂ | Ph | Et | CH₂Ph-4-SO₂Me | |
| 123 | N(CH₂CH₃)(CH₂)₂CH₃ | Ph | Et | CH₂Ph-4-SO₂Me | |
| 124 | NH(5-OH-1H-pyrazol-3-yl) | Ph | Et | CH₂Ph-4-SO₂Me | |
| 125 | NH(1,3,5-triazin-2-yl) | Ph | Et | CH₂Ph-4-SO₂Me | |
| 126 | NHCH₂CH(CH₃)(CH₂)₂CH₃ | Ph | Et | CH₂Ph-4-SO₂Me | 586 |
| 127 | NH(CH₂)₂OCH₂CH₃ | Ph | Et | CH₂Ph-4-SO₂Me | 574 |
| 128 | NHCH₂cyclopropyl | Ph | Et | CH₂Ph-4-SO₂Me | 556 |
| 129 | NH(isoxazol-3-yl) | Ph | Et | CH₂Ph-4-SO₂Me | |
| 130 | NH(6-OH-pyridin-2-yl) | Ph | Et | CH₂Ph-4-SO₂Me | 595 |
| 131 | NH(CH₂)₃SCH₃ | Ph | Et | CH₂Ph-4-SO₂Me | |
| 132 | N(CH₃)C(CH₃)₃ | Ph | Et | CH₂Ph-4-SO₂Me | |
| 133 | N(CH₃)CH(CH₃)CH₂CH₃ | Ph | Et | CH₂Ph-4-SO₂Me | 572 |
| 134 | NHCH₂C(═CH₂)CH₃ | Ph | Et | CH₂Ph-4-SO₂Me | 556 |
| 135 | NHCH₂CH(OH)CH₂CH₃ | Ph | Et | CH₂Ph-4-SO₂Me | 574 |
| 136 | NHCH₂C(CH₃)₂CH₂OH | Ph | Et | CH₂Ph-4-SO₂Me | 588 |
| 137 | NHCH(CH₂OH)CH(CH₃)₂ | Ph | Et | CH₂Ph-4-SO₂Me | 588 |
| 138 | (R)-2-CH₂OH-pyrrolidin-1-yl | Ph | Et | CH₂Ph-4-SO₂Me | 586 |
| 138 | NH(3-oxo-isoxazolidin-4-yl) | Ph | Et | CH₂Ph-4-SO₂Me | |
| 140 | NHCH(CH₃)CH₂OH | Ph | Et | CH₂Ph-4-SO₂Me | 560 |
| 141 | NHCH(CH₃)CH₂CH₃ | Ph | Et | CH₂Ph-4-SO₂Me | 558 |
| 142 | NHCH(CH₂OH)CH₂CH₃ | Ph | Et | CH₂Ph-4-SO₂Me | 574 |
| 143 | (R)-NHCH₂CH(OH)CH₃ | Ph | Et | CH₂Ph-4-SO₂Me | 560 |
| 144 | (S)-NHCH₂CH(OH)CH₃ | Ph | Et | CH₂Ph-4-SO₂Me | 560 |
| 145 | NH(1-CH₃-pyrazol-5-yl) | Ph | Et | CH₂Ph-4-SO₂Me | |
| 146 | NH(1H-5-CH₃-pyrazol-3-yl) | Ph | Et | CH₂Ph-4-SO₂Me | |
| 147 | NHCH₂CN | Ph | Et | CH₂Ph-4-SO₂Me | |
| 148 | NH(4-CH₃-oxazol-2-yl) | Ph | Et | CH₂Ph-4-SO₂Me | |
| 149 | N(CH₃)(CH₂)₂OCH₃ | Ph | Et | CH₂Ph-4-SO₂Me | 574 |
| 150 | NH(1H-tetrazol-5-yl) | Ph | Et | CH₂Ph-4-SO₂Me | |
| 151 | NHCH₂C(O)NHOH | Ph | Et | CH₂Ph-4-SO₂Me | 575 |
| 152 | (S)-NHCH₂CH(OH)CH₂OH | Ph | Et | CH₂Ph-4-SO₂Me | 576 |
| 153 | (R)-NHCH₂CH(OH)CH₂OH | Ph | Et | CH₂Ph-4-SO₂Me | 576 |
| 154 | NHC(CH₃)₂(CH₂)₂OH | Ph | Et | CH₂Ph-4-SO₂Me | 588 |
| 155 | NHCH₂C(CH₃)₂OCH₃ | Ph | Et | CH₂Ph-4-SO₂Me | 588 |
| 156 | NHCH(CH₂OH)CH(CH₃)₂ | Ph | Et | CH₂Ph-4-SO₂Me | |
| 157 | 2-azabicyclo[2.2.1]heptan-2-yl | Ph | Et | CH₂Ph-4-SO₂Me | 582 |
| 158 | NHCH₂(1H-imidazol-2-yl) | Ph | Et | CH₂Ph-4-SO₂Me | 582 |
| 159 | (S)-3-CH₃-piperazin-1-yl | Ph | Et | CH₂Ph-4-SO₂Me | |
| 160 | (R)-3-CH₃-piperazin-1-yl | Ph | Et | CH₂Ph-4-SO₂Me | 585 |
| 161 | (R)-NHCH₂(tetrahydrofuran-2- | Ph | Et | CH₂Ph-4-SO₂Me | 586 |
| | yl) | | | | |
| 162 | N(CH₃)(pyridin-4-yl) | Ph | Et | CH₂Ph-4-SO₂Me | |
| 163 | N(CH₃)C(CH₃)₂CN | Ph | Et | CH₂Ph-4-SO₂Me | |
| 164 | NH(CH₂)₂CH(OH)CH₃ | Ph | Et | CH₂Ph-4-SO₂Me | 574 |
| 165 | NH(4-CN-isoxazol-3-yl) | Ph | Et | CH₂Ph-4-SO₂Me | |
| 166 | NH(3-CH₃-cyclopentyl) | Ph | Et | CH₂Ph-4-SO₂Me | 584 |
| 167 | (S)-3-OH-pyrrolidin-1-yl | Ph | Et | CH₂Ph-4-SO₂Me | |
| 168 | NH(1H-3-CH₃-pyrazol-5-yl) | Ph | Et | CH₂Ph-4-SO₂Me | |
| 169 | NHCH(CH₃)CH₂N(CH₃)₂ | Ph | Et | CH₂Ph-4-SO₂Me | |
| 170 | NHCH₂CH(CH₃)N(CH₃)₂ | Ph | Et | CH₂Ph-4-SO₂Me | |
| 171 | (S)-NHCH₂(tetrahydrofuran-2- | Ph | Et | CH₂Ph-4-SO₂Me | |
| | yl) | | | | |
| 172 | C(CH₃)₃ | Ph | Et | CH₂Ph-4-SO₂Me | 543 |

TABLE II
All compounds in Table II are of formula (Ib) below.

| | |
|---|---|
| | (Ib) |
| 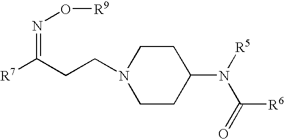 | |

| Compound | | | | | LCMS |
|---|---|---|---|---|---|
| No. | R⁹ | R⁷ | R⁵ | R⁶ | (MH+) |
| 1 | allyl | Ph | Et | CH₂Ph-4-SO₂Me | 512 |
| 2 | ethyl | Ph | Et | CH₂Ph-4-SO₂Me | 500 |

Ph = phenyl; Et = ethyl

The compounds of formula (I), (Ia) and (Ib), where R², R^{2a}, R⁴ and R^{4a} and, if present, R³ and R^{3a} are all hydrogen, can be prepared as shown in Schemes 1 and 2 below, or by adaptation of known methods described in the art. Compounds wherein one or more of R², R^{2a}, R⁴ and R^{4a} and, if present, R³ and R^{3a} are hydrogen can be prepared by methods analogous to those shown in Schemes 1 and 2, by changing one or more reactants in the methods of Schemes 1 or 2, or by adaptation of known methods described in the art. In a further aspect the invention provides processes for preparing the compounds of formula (I), (Ia) and (Ib). Many of the intermediates in the processes are novel and these are provided as further features of the invention. Compounds of formula (I) wherein R¹ is CHR⁷S(O)₂NR¹⁰R¹¹ can be made by routine adaptation of methods herein described combined with methods described in the literature.

Thus, a compound of formula (I) wherein R¹ is CHR⁷OC(O)R⁸ can be prepared by reacting a compound of formula (II):

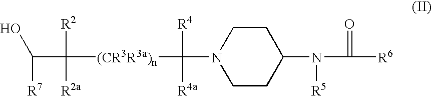

with a compound of formula R⁸C(O)Cl in the presence of a suitable base (such as a tertiary amine, for example of formula R^{a}R^{b}R^{c}N, where R^{a}, R^{b} and R^{c} are, independently, C₁₋₆ alkyl; for example triethylamine) and in a suitable solvent (such as dichloromethane) at a temperature in the range 10-50° C. Alternatively, a compound of formula (II) is pre-treated with sodium hydride in a suitable solvent (for example N-methylpyrrolidone) and the compound of formula R⁸C(O)Cl added to this mixture.

A compound of formula (I) wherein R¹ is CR⁷═NOR⁹ can be prepared by reacting a compound of formula (III):

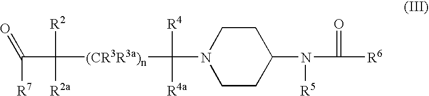

(as a free base or in salt form, for example, in the form of a hydrochloride) with a compound of formula R⁹ONH₂ (preferably in salt form, for example in the form of a hydrochloride) in a suitable solvent (such as dichloromethane) at a temperature in the range 10-50° C.

A compound of formula (I) wherein R¹ is CHR⁷OC(O)NHR¹³ can be prepared by reacting a compound of formula (II) with a compound of formula R¹³NCO.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)). Examples of these conditions are:

(1) (the respiratory tract) obstructive diseases of airways including: chronic obstructive pulmonary disease (COPD) (such as irreversible COPD), pulmonary fibrosis; asthma (such as bronchial, allergic, intrinsic, extrinsic or dust asthma, particularly chronic or inveterate asthma (for example late asthma or airways hyper-responsiveness)); bronchitis (such as eosinophilic bronchitis); acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, inhibiting the entry of viruses into target cells, Acquired Immunodeficiency Syndrome (ADS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura, disorders of the menstrual cycle, glomerulonephritis or cerebral malaria.

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia) or (Ib), or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (especially CCR5 receptor activity) in a warm blooded animal, such as man, in need of such treatment, which comprises administering to said animal an effective amount of a compound of the present invention, or a pharmaceutically acceptable salt thereof or a solvate thereof.

The present invention further provides a method of treating a chemokine mediated disease state (especially a CCR5 mediated disease state, such as rheumatoid arthritis) in a warm blooded animal (such as man) suffering from, or at risk of, said disease, which comprises administering to an animal in need of such treatment a therapeutically effective amount of a compound of formula (I), (Ia) or (Ib), or a pharmaceutically acceptable salt thereof or solvate thereof.

The invention also provides a compound of the formula (I), (Ia) or (Ib), or a pharmaceutically acceptable salt thereof or a solvate thereof, for use in therapy (including prophylaxis); for example in the treatment of a chemokine mediated disease state (especially a CCR5 mediated disease state) in a warm blooded animal, such as man, such as in the treatment of rheumatoid arthritis.

The invention also provides a compound of the formula (I):

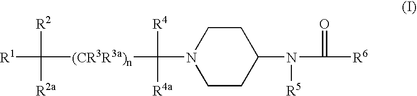

wherein: R¹ is a group selected from:

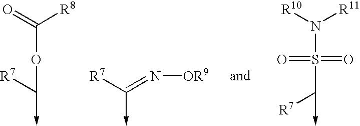

R², R^{2a}, R⁴ and R^{4a} are, independently, hydrogen or C₁₋₄ alkyl;

R³ and R^{3a} are, independently, hydrogen, C₁₋₄alkyl or C₁₋₄ alkoxy;

n is 0 or 1;

R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;

R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;

R⁷ is phenyl, heteroaryl, phenyl(C₁₋₄ alkyl) or heteroaryl(C₁₋₄ alkyl);

R⁸ is C₁₋₈ alkyl, OR¹², NR¹³R¹⁴, phenyl, heteroaryl, phenyl(C₁₋₂)alkyl or heteroaryl(C₁₋₂)alkyl;

R⁹, R¹⁰ and R¹¹ are, independently, hydrogen, C₁₋₆ alkyl (optionally substituted by C₁₋₆ alkoxy, phenyl or heteroaryl), phenyl or heteroaryl; or R¹⁰ and R¹¹ may join to form a 5- or 6-membered ring which may additionally include an oxygen atom or a further nitrogen atom, said ring being optionally substituted with C₁₋₄ alkyl, C(O)H or C(O)(C₁₋₄ alkyl);

R¹² and R¹³ are C₁₋₈ alkyl (optionally substituted by halogen, OH, cyano, C₁₋₆ alkoxy, C₁₋₆ hydroxyalkoxy, C₁₋₆ alkylthio, C₃₋₆ cycloalkyl, NR¹⁵R¹⁶, C(O)NH(OH), NHC(O)(C₁₋₄ alkyl), heterocyclyl, phenyl or heteroaryl), C₃₋₆ alkenyl, C₃₋₆ alkynyl, C₃₋₆ cycloalkyl (optionally substituted by C₁₋₆ alkyl), phenyl, heteroaryl or heterocyclyl;

R¹⁴ is hydrogen or is independently selected from the list of options recited for R¹³;

or R¹³ and R¹⁴ join to form a 5, 6, 7 or 8-membered monocyclic or bicyclic ring system which is optionally unsaturated, optionally includes a further nitrogen atom or also includes an oxygen or sulphur atom, and which is optionally substituted by OH, C₁₋₆ alkyl or C₁₋₆ hydroxyalkyl;

R¹⁵ and R¹⁶ are, independently, hydrogen or C₁₋₆ alkyl;

wherein the phenyl, heteroaryl and heterocyclyl rings of any of the foregoing are independently optionally substituted by halo, cyano, nitro, oxo, hydroxy, C₁₋₄ alkyl, C₁₋₄ hydroxyalkyl, C₁₋₄ alkoxy, S(O)ₘC₁₋₄ alkyl, S(O)₂NR¹⁷R¹⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;

R¹⁷ and R¹⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);

m, p and q are, independently, 0, 1 or 2;

or a pharmaceutically acceptable salt thereof or a solvate thereof; for use as a medicament, especially a medicament for the treatment of rheumatoid arthritis.

The invention further provides a compound of the formula (Ia), or a pharmaceutically acceptable salt thereof or a solvate thereof, for use as a medicament, especially a medicament for the treatment of rheumatoid arthritis.

In a further aspect the invention provides a compound of formula (Ib) wherein R⁵, R⁶, R⁷ and R⁹ are as defined immediately above, or a pharmaceutically acceptable salt thereof or a solvate thereof, for use as a medicament, especially a medicament for the treatment of rheumatoid arthritis.

In another aspect the present invention provides the use of a compound of the formula (I), (Ia) or (Ib), or a pharmaceutically acceptable salt thereof or a solvate thereof, in the manufacture of a medicament for use in therapy (for example in modulating chemokine receptor activity (especially CCR5 receptor activity (especially in the treatment of rheumatoid arthritis)) in a warm blooded animal, such as man).

The invention further provides the use of a compound of formula (I), (Ia) or (Ib), or a pharmaceutically acceptable salt thereof, in the manufacture of a medicament for use in the treatment of:

(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastroenteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle;

in a warm blooded animal, such as man.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia) and (Ib), or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), (Ia) and (Ib), or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph.Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1.0 |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Clamorgan, UK. Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.

(iv) in general, the course of reactions was followed by TLC and reaction times are given for illustration only;

(v) yields, when given, are-for illustration only and are-not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:

| | |
|---|---|
| DMSO | dimethyl sulphoxide; |
| DMF | N-dimethylformamide; |
| DCM | dichloromethane; |
| DIPEA | N,N-diisopropylethylamine; |
| NMP | N-methylpyrrolidinone; |
| HATU | O-(7-Azabenzotriazol-1-yl)-N,N,N′,N′-tetramethyluronium |
| | hexafluorophosphate; |
| THF | tetrahydrofuran; |
| EtOH | ethanol; and |
| EtOAc | ethyl acetate. |

### EXAMPLE 1

This Example illustrates the preparation of N-[1-(3-[4-fluorophenyl]-3-[1-pyrrolidinylcarbonyloxy]propyl)-4-piperidinyl]-N-ethyl-4-fluorophenylacetamide (Compound No. 1 of Table I).

To a stirred solution of N-[1-(3-[4-fluorophenyl]-3-hydroxypropyl)-4-piperidinyl]-N-ethyl-4-fluorophenylacetamide (Method 1) (101 mg, 0.24 mmol) in NMP (2 mL) was added sodium hydride (20 mg 60% dispersion, 0.48 mmol) and the resulting mixture was stirred at room temperature for 10 min. 1-Pyrrolidinecarbonyl chloride (35 mg, 0.26 mmol) was added and the resulting mixture stirred at room temperature for 20 h. The mixture was partitioned between water and ethyl acetate. The aqueous phase was evaporated and the residue triturated with diethyl ether to yield the title compound as a solid (110 mg, 89%); NMR (DMSO at 373K): 1.09 (t, 3H), 1.47 (m, 2H), 1.71 (m, 2H), 1.75-3.05 (m, 6H), 3.32 (m, 12H), 3.70 (s, 2H), 3.84 (br m, 1H), 5.68 (t, 1H), 7.10 (m, 4H), 7.27 (m, 2H) and 7.39 (m, 2H); MS: 514.

The same method was used for Compound No. 3 of Table I:

NMR (CDCl₃): 0.95-1.25 (br m, 9H), 1.80 (br m, 2H), 2.01 (m, 2H), 2.26 (m, 2H), 2.94 (br m, 2H), 3.26 (m, 6H), 3.37 (t, 2H), 3.50 and 4.40 (m, 1H), 3.66 and 3.69 (s, 2H), 5.70 (m, 1H), 7.00 (m, 4H) and 7.25 (m, 4H); MS: 516.

### EXAMPLE 2

This Example illustrates the preparation of N-[1-(3-[4-fluorophenyl]-3-methoxycarbonyloxypropyl)-4-piperidinyl]-N-ethyl4-methanesulfonyl-phenylacetamide hydrochloride (Compound No. 2 of Table I).

To a solution of N-[1-(3-[4-fluorophenyl]-3-hydroxypropyl)-4-piperidinyl]-N-ethyl4-methanesulfonylphenylacetamide (Method 11) (230 mg, 0.48 mmol) in DCM (5 mL) was added 4-nitrophenylchloroformate (101 mg, 0.50 mmol) and triethylamine (80 μL, 0.50 mmol) and the resulting mixture stirred at room temperature for 30 min. The mixture applied to an ISOLUTE™ SCX column (10 g) which was then washed with DCM followed by MeOH. The crude product was triturated with ethereal HCl/iso-hexane to yield the title compound as a solid (180 mg, 70%); NMR (CDCl₃): 0.85 (m, 3H), 1.05-1.95 (m, 6H), 2.02 (m, 1H), 2.16 (m, 1H), 2.30 (m, 2H), 2.83 (m, 1H), 2.96 (m, 1H), 3.02 (s, 3H), 3.32 (q, 2H), 3.50 and 4.28 (m, 1H), 3.72 (s, 3H), 3.77 and 3.79 (2s, 2H), 5.62 (t, 3H), 7.03 (m, 2H), 7.32 (m, 2H), 7.45 (m, 2H) and 7.90 (d, 2H); MS: 535.

### EXAMPLE 3

This Example illustrates the preparation of N-[1-(3-phenyl-3-[2-pyrrolidin-1-ylethylaminocarbonyloxy]propyl)-4-piperidinyl]-N-ethyl-4-methanesulfonylphenylacetamide (Compound No. 4 of Table I).

A solution of N-[1-(3-phenyl-3-[4-nitrophenoxycarbonyloxy]propyl)₄-piperidinyl]-N-ethyl-4-methanesulfonylphenylacetamide (Method 3) (0.25 g, 0.40 mmol) in DCM (2 mL) was added to 1-(2-aminoethyl)pyrrolidine (127 μL, 1.0 mmol) and the resulting mixture stirred at room temperature for 16 h. The mixture was partitioned between DCM (5 mL) and saturated aqueous sodium bicarbonate solution (5 mL). The organic phase was applied to an ISOLUTE™ SCX column (10 g) which was then washed with DCM followed by MeOH followed by 4% ammonia/MeOH to give the title compound (180 mg, 75%); NMR (CDCl₃): 0.86 and 0.88 (t, 3H), 1.15 (m, 2H), 1.25 (m, 2H), 1.30-2.20 (m, 8H), 2.32 (m, 2H), 2.49 (m, 4H), 2.57 (m, 2H), 2.87 (m, 1H), 2.97 (m, 1H), 3.03 (s, 3H), 3.29 (m, 4H), 3.50 and 4.20 (m, 1H), 3.78 and 3.79 (s, 2H), 5.22 (m, 1H), 5.71 (m, 1H), 7.28 (m, 1H), 7.33 (m, 4H), 7.44 (d, 2H) and 7.90 (d, 2H); MS: 599.

The same method was used for Compound No. 117 of Table I:

NMR (DMSO at 373K): 1.14 (t, 3H), 1.38 (m, 2H), 1.53 (m, 2H), 1.60-2.10 (m, 8H), 2.33 (m, 2H), 2.75-3.05 (m, 6H), 3.15 (s, 3H), 3.31 (m, 2H), 3.47 (br m, 1H), 3.65 (m, 1H), 3.81 (m, 1H), 3.83 (s, 2H), 4.47 (br m, 1H), 5.68 (m, 1H), 7.28 (m, 1H), 7.33 (m, 4H), 7.51 (d, 2H) and 7.85 (d, 2H); MS: 586.

The same method was used for Compound No. 136 of Table I:

NMR (CDCl₃): 0.86 and 0.93 (2s, 6H), 1.16 and 1.27 (2t, 3H), 1.35-2.20 (m, 9H), 2.32 (q, 2H), 2.80-3.50 (m, 11H), 3.78 and 3.80 (2s, 2H), 4.40 (m, 1H), 5.05 (m, 1H), 5.70 (m, 1H), 7.32 (m, 5H), 7.46 (m, 2H) and 7.91 (m, 2H); MS: 588.

### EXAMPLE 4

This Example illustrates the preparation of N-[1-(3-phenyl-3-[tert-butylcarbonyloxy]propyl)-4-piperidinyl)-N-ethyl-4-methanesulfonylphenylacetamide (Compound No. 172 of Table I).

To a solution of N-[1-(3-phenyl-3-hydroxypropyl)-4-piperidinyl]-N-ethyl-4-methanesulfonylphenylacetamide (Method 4) (150 mg, 0.33 mmol) in DCM (2 mL) was added trimethylacetyl chloride (44 μL, 0.36 mmol) and triethylamine (50 μL, 0.36 mmol) and the resulting mixture was stirred at 40° C. for 16 h then allowed to cool. The mixture was partitioned between DCM and water, the organic phase was washed with brine, dried (Na₂SO₄) and evaporated giving the title compound (141 mg, 79%); NMR: 1.02 and 1.15 (t, 3H), 1.16 (s, 9H), 1.46 (m, 2H), 1.66 (m, 2H), 1.89 (m, 2H), 2.26 (m, 2H), 2.83 (m, 2H), 3.20 (m, 4H), 3.30 (m, 6H), 3.65 and 4.09 (m, 1H), 3.81 and 3.87 (2s, 2H), 5.69(m, 1H), 7.33 (m, 5H), 7.59 (d, 2H) and 7.85 (d, 2H); MS: 543.

### EXAMPLE 5

This Example illustrates the preparation of N-[1-(3-phenyl-3-allyloxyiminopropyl)-4-piperidinyl]-N-ethyl4-methanesulfonylphenylacetamide (Compound No. 1 of Table II).

To a solution of N-[1-(3-phenyl-3-oxopropyl)-4-piperidinyl]-N-ethyl-4-methanesulfonylphenylacetamide hydrochloride (Method 5) (500 mg, 1 mmol) in DCM (10 mL) was-added O-allyl hydroxylamine hydrochloride (131 mg, 1.2 mmol) followed by sodium sulphate (2 g). The resulting mixture was stirred at reflux for 20 h then allowed to cool. The mixture was filtered and the filtrate diluted with DCM, washed with water and brine, dried (Na₂SO₄) and concentrated to afford the title compound as a solid (400 mg, 78%);

NMR (CDCl₃): 1.29 (m, 3H), 1.87 (m, 2H), 2.64 (m, 2H), 2.82 (m, 2H), 3.03 (s, 3H), 3.14 (m, 1H), 3.41 (m, 2H), 3.65 (m, 2H), 3.78 (s, 2H), 4.70 (d, 2H), 5.27 (m, 2H), 5.29 (s, 2H), 6.00 (m, 1H), 7.41 (m, 5H), 7.74 (m, 2H) and 7.88 (m, 2H); MS: 512.

Methods

Method 1

Preparation of N-[1-(3-[4-fluorophenyl]-3-hydroxypropyl)-4-piperidinyl]-N-ethyl4-fluorophenylacetamide

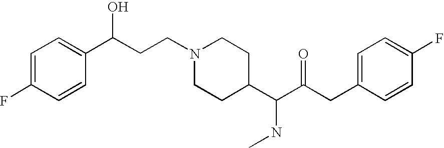

To a solution of N-[1-(3-[4-fluorophenyl]-3-oxopropyl)-4-piperidinyl]-N-ethyl4-fluorophenylacetamide (Method 2) (8.7 mmol) in methanol (100 mL) was added sodium borohydride (0.66 g, 17.4 mmol). The resulting mixture was stirred at room temperature for 20 h. Water (5 mL) was added and the mixture evaporated. The residue was purified by silica column chromatography (gradient elution from ethyl acetate to 50% ethyl acetate/MeOH) to give the title compound (2.77 g, 76%): NMR (CDCl₃): 0.85 and 1.12 (t, 3H), 1.21 (m, 2H), 1.45 (m, 1H), 1.55-1.90 (m, 3H), 1.98 (m, 1H), 2.22 (m, 1H), 2.56 (m, 1H), 2.63 (m, 1H), 3.12 (m, 2H), 3.27 (q, 2H), 3.58 and 4.49 (m, 1H), 3.67 and 3.71 (s, 2H), 4.88 (m, 1H), 7.00 (m, 4H), 7.22 (m, 2H), 7.31 (m, 2H); MS: 417.

Method 2

Preparation of N-[1-(3-[4-fluorophenyl]-3-oxopropyl)-4-piperidinyl]-N-ethyl4-fluorophenylacetamide

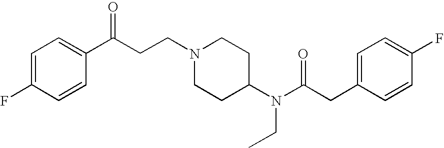

To a solution of N-(4-piperidinyl)-N-ethyl4-fluorophenylacetamide (Method 9) (2.3 g, 8.7 mmol) in DMF (50 mL) was added DIPEA (3 mL, 17.4 mmol) and 3-chloro-4′-fluoropropiophenone (1.7 g, 9.1 mmol). The resulting mixture was stiffed at room temperature for 16 h then partitioned between water and ethyl acetate. The organic phase was washed with brine, dried (Na₂SO₄) and evaporated to give the title compound as an oil (˜4 g) which was used in the next reaction without further purification; MS: 415.

Method 3

Preparation of N-[1-(3-phenyl-3-[4-nitrophenoxycarbonyloxy]propyl)-4-piperidinyl]-N-ethyl4-methanesulfonylphenylacetamide

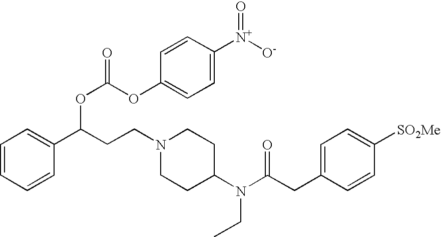

To a solution of N-[1-(3-phenyl-3-hydroxypropyl)-4-piperidinyl]-N-ethyl-4-methanesulfonylphenylacetamide (Method 4) (0.50 g, 1.1 mmol) in DCM (5 mL) was added 4-nitrophenylchloroformate (240 mg, 1.2 mmol) followed by triethylamine (167 μL, 1.2 mmol). The resulting mixture was stirred at room temperature for 24 h then partitioned between DCM and saturated aqueous sodium bicarbonate solution. The organic phase was washed with brine, dried (Na₂SO₄) and evaporated giving the title compound as a gum (645 mg, 95%) which was characterised by LC-MS; MS: 624.

Method 4

Preparation of N-[1-(3-phenyl-3-hydroxypropyl)-4-piperidinyl]-N-ethyl4-methanesulfonylphenylacetamide

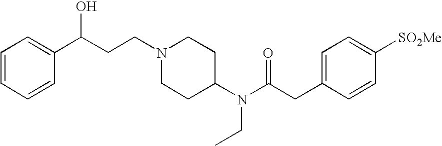

To a solution of N-[1-(3-phenyl-3-oxopropyl)-4-piperidinyl]-N-ethyl-4-methanesulfonylphenylacetamide hydrochloride (Method 5) (5.00 g, 10.1 mmol) in methanol (150 mL) was added sodium borohydride (0.96 g, 25.4 mmol) portionwise. The resulting mixture was stirred at room temperature for 20 h. Water (10 mL) was added and the mixture was evaporated. The residue was purified by silica column chromatography (gradient elution from ethyl acetate to 50% ethyl acetate/MeOH) to give the title compound (3.92 g, 84%);

NMR: (CDCl₃): 1.14 and 1.23 (t, 3H), 1.56 (m, 1H), 1.75 (m, 2H), 1.83 (m, 3H), 1.98 (m, 1H), 2.20 (m, 1H), 2.56 (m, 1H), 2.66 (m, 1H), 3.02 (s, 3H), 3.10 (m, 1H), 3.18 (m, 1H), 3.31 (q, 2H), 3.57 and 4.49 (m, 1H), 3.79 and 3.80 (s, 2H), 4.94 (m, 1H), 7.23 (m, 1H), 7.34 (m, 4H), 7.44 (d, 2H) and 7.90 (d, 2H); MS: 459.

Method 5

Preparation of N-[1-(3-phenyl]-3-oxopropyl)-4-piperidinyl]-N-ethyl4-methanesulfonylphenylacetamide hydrochloride

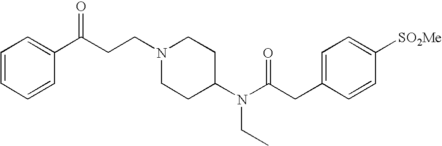

To a solution of N-(4-piperidinyl)-N-ethyl-4-methanesulfonylphenylacetamide (Method 6) (14.8 g, 45.8 mmol) and DIPEA (24 mL, 137 mmol) in DMP (250 mL) was added 3-chloropropiophenone (7.3 g, 43.5 mmol). The resulting mixture was stirred at room temperature for 20 h. The mixture was evaporated and the residue triturated with 5% MeOH/EtOAc to give a solid which was collected by filtration and washed with EtOAc affording the title compound (16.9 g, 75%); NMR (DMSO at 373K): 1.14 (t, 3H), 1.77 (m, 2H), 2.34 (m, 2H), 3.11 (m, 2H), 3.15 (s, 3H), 3.45-3.60 (m, 6H), 3.65 (t, 2H), 3.93 (s, 2H), 4.25 (br m, 1H), 7.53 (m, 4H), 7.65 (m, 1H), 7.84 (d, 2H) and 7.98 (d, 2H); MS: 457.

Method 6

Preparation of N-(4-piperidinyl)-N-ethyl-4-methanesulfonylphenylacetamide

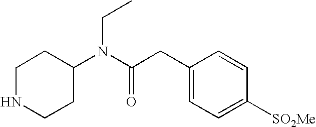

To a solution of N-(1-phenylmethyl-4-piperidinyl)-N-ethyl-4-methanesulfonylphenylacetamide (Method 7) (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the title compound (24.9 g, 94%); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H); MS: 325.

Method 7

Preparation of N-(1-phenylmethyl-4-piperidinyl)-N-ethyl-4-methanesulfonyl-phenylacetamide

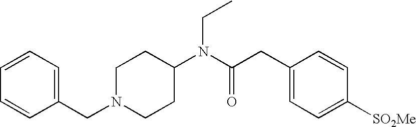

To a solution of 1-phenylmethyl-4-ethylaminopiperidine dihydrochloride (Method 8) (32.0 g, 110 mmol) in DCM (500 mL) was DIPEA (60 mL) with stirring to ensure complete dissolution. 4-Methanesulfonylphenylacetic acid (25.0 g, 117 mmol), 4-dimethylamino-pyridine (2.0 g) and dicyclohexylcarbodiimide (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent 10% MeOH/ethyl acetate) to afford the title compound (35 g, 76%);

NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415.

Method 8

Preparation of Phenylmethyl-4-ethylaminopiperidine Dihydrochloride

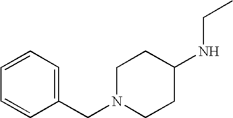

To a solution of 1-phenylmethyl-4-piperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (500 mL) and concentrated hydrochloric acid (20 mL) was added. The resulting crystals were collected, washed with diethyl ether and dried giving the title compound as a solid (38 g); NMR: (CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219.

Method 9

Preparation of N-4-piperidinyl-N-ethyl-4-fluorophenylacetamide

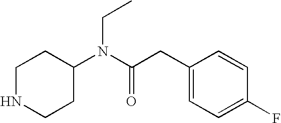

This was prepared by reacting N-(1-phenylmethyl-4-piperidinyl-N-ethyl4-fluorophenylacetamide according to the procedure used for Method 6; NMR (formic acid salt): 0.97 and 1.10 (t, 3H), 1.46 and 1.62 (m, 2H), 1.8-2.0 (m, 2H), 2.78 (m, 2H), 3.1-3.3 (m, 4H), 3.65 and 3.74 (s, 2H), 3.97 and 4.22 (m, 1H), 7.08 (m, 2H), 7.25 (m, 2H), 8.42 (s, 1H); MS: 265.

Method 10

Preparation of N-(1-phenylmethyl-4-piperidinyl-N-ethyl-4-fluorophenylacetamide

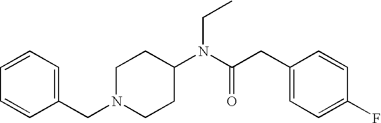

This was prepared by reacting 1-phenylmethyl-4-ethylaminopiperidine dihydrochloride with 4-fluorophenylacetic acid according to the procedure used for Method 7; NMR (CDCl₃): 1.13 and 1.19 (t, 3H), 1.35 and 1.85 (m, 2H), 1.74 and 2.08 (m, 2H), 2.90 (br m, 2H), 3.30 (m, 2H), 3.46 (s, 2H), 3.66 (s, 2H), 3.55 and 4.42 (m, 1H), 7.00 (m, 2H), 7.2-7.3 (m, 7H); MS: 355.

Method 11

Preparation of N-[1-(3-[4-fluorophenyl]-3-hydroxypropyl)-4-piperidinyl]-N-ethyl-4-methanesulfonylphenylacetamide

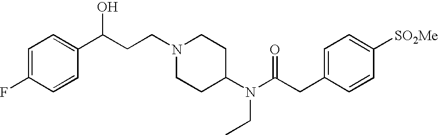

This was prepared by reacting N-[1-(3-[4-fluorophenyl]-3-oxopropyl)-4-piperidinyl]-N-ethyl4-methanesulfonylphenylacetamide hydrochloride (Method 12) according to the procedure used for Method 4; NMR: MS: 477.

Method 12

Preparation of N-[1-(3-[4-fluorophenyl]-3-oxopropyl)-4-piperidinyl]-N-ethyl-4-methanesulfonylphenylacetamide hydrochloride

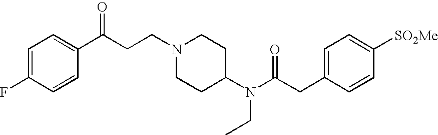

To a solution of N-4-piperidinyl-N-ethyl-4-methanesulfonylphenylacetamide (Method 6) (1.3 g, 4.0 mmol) in DMF (25 mL) was added DIPEA (2 mL, 11.5 mmol) and 3-chloro-4′-fluoropropiophenone (770 mg, 4.0 mmol). The resulting mixture was stirred at room temperature overnight then evaporated. The residue was heated to reflux with 5% methanol in ethyl acetate giving a white solid which was isolated (1.6 g, 80%). NMR: 1.00 and 1.16 (t, 3H), 1.75 (t, 2H), 2.23 (q, 2H), 3.10 (t, 2H), 3.18 (s, 3H), 3.30 (m, 2H), 3.35 and 3.64 (q, 2H), 3.56 (m, 2H), 3.82 and 3.93 (s, 2H), 4.15 and 4.28 (m, 1H), 7.40 (m, 2H), 7.50 (m, 2H), 7.83 (m, 2H), 8.07 (m, 2H); MS: 475.

### EXAMPLE 6

The ability of compounds to inhibit the binding of RANTES or MP-1α was assessed by an in vitro radioligand binding assay. Membranes were prepared from Chinese hamster ovary cells which expressed the recombinant human CCR5 receptor. These membranes were incubated with 0.1 nM iodinated RANTES or MIP-1α, scintillation proximity beads and various concentrations of the compounds of the invention in 96-well plates. The amount of iodinated RANTES or MIP-1α bound to the receptor was determined by scintillation counting. Competition curves were obtained for compounds and the concentration of compound which displaced 50% of bound iodinated RANTES or MP-1α was calculated (IC₅₀). Certain compounds of formula (I) had an IC₅₀ of less than 50 μM.

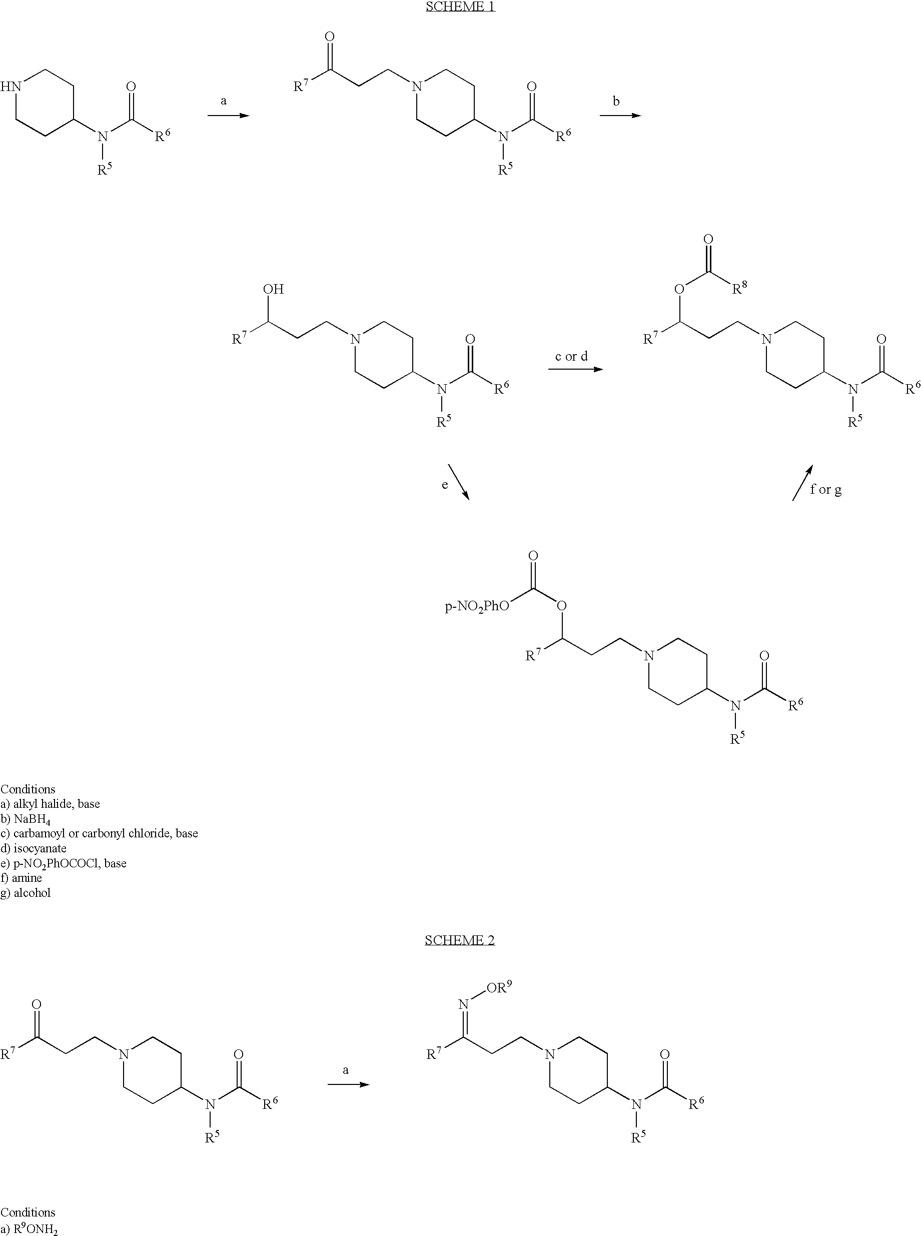

1. A compound of formula (I): 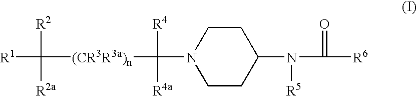
wherein:
R¹ is a group selected from: 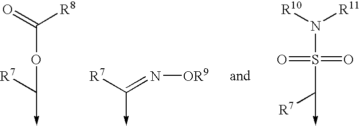
R², R^{2a}, R⁴ and R^{4a} are, independently, hydrogen or C₁₋₄ alkyl;
R³ and R^{3a} are, independently, hydrogen, C₁₋₄ alkyl or C₁₋₄ alkoxy;
n is 0 or 1;
R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;
R⁷ is phenyl, heteroaryl, phenyl(C₁₋₄ alkyl) or heteroaryl(C₁₋₄ alkyl);
R⁸ is C₁₋₈ alkyl, OR¹², NR¹³R¹⁴, phenyl, heteroaryl, phenyl(C₁₋₂)alkyl or heteroaryl(C₁₋₂)alkyl;
R⁹, R¹⁰ and R¹¹ are, independently, hydrogen, C₁₋₆ alkyl (optionally substituted by C₁₋₆ alkoxy, phenyl or heteroaryl), phenyl or heteroaryl; or R¹⁰ and R¹¹ may join to form a 5- or 6-membered ring which may additionally include an oxygen atom or a further nitrogen atom, said ring being optionally substituted with C₁₋₄ alkyl, C(O)H or C(O)(C₁₋₄ alkyl);
R¹² and R¹³ are C₁₋₈ alkyl (optionally substituted by halogen, OH, cyano, C₁₋₆ alkoxy, C₁₋₆ hydroxyalkoxy, C₁₋₆ alkylthio, C₃₋₆ cycloalkyl, NR¹⁵R¹⁶, C(O)NH(OH), NHC(O)(C₁₋₄ alkyl), heterocyclyl, phenyl or heteroaryl), C₃₋₆ alkenyl, C₃₋₆ alkynyl, C₃₋₆ cycloalkyl (optionally substituted by C₁₋₆ alkyl), phenyl, heteroaryl or heterocyclyl;
R¹⁴ is hydrogen or is independently selected from the list of options recited for R¹³;
or R¹³ and R¹⁴ join to form a 5, 6, 7 or 8-membered monocyclic or bicyclic ring system which is optionally unsaturated, optionally includes a further nitrogen atom or also includes an oxygen or sulphur atom, and which is optionally substituted by OH, C₁₋₆ alkyl or C₁₋₆ hydroxyalkyl;
R¹⁵ and R¹⁶ are, independently, hydrogen or C₁₋₆ alkyl;
wherein the phenyl, heteroaryl and heterocyclyl rings of any of the foregoing are independently optionally substituted by halo, cyano, nitro, oxo, hydroxy, C₁₋₄ alkyl, C₁₋₄ hydroxyalkyl, C₁₋₄ alkoxy, S(O)ₘC₁₋₄ alkyl, S(O)₂NR¹⁷R¹⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R¹⁷ and R¹⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, p and q are, independently, 0, 1 or 2;
or a pharmaceutically acceptable salt thereof or a solvate thereof;
provided that when R¹ is 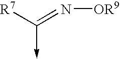
n is 0 or 1; R², R^{2a}, R³, R^{3a}, R⁴, R^{4a}, R⁵ and R⁹ are all hydrogen; and R⁶ is unsubstituted phenyl; then R⁷ is not optionally substituted phenyl, or a salt thereof.

2. A compound of formula (I) as claimed in claim 1 wherein n is 0; R², R^{2a} and R⁴ are all hydrogen; and R^{4a} is hydrogen or methyl.

3. A compound of formula (I) as claimed in claim 1 wherein n is 1; R², R^{2a}, R³, R^{3a} and R⁴ are all hydrogen; and R^{4a} is hydrogen or methyl.

4. A compound as claimed in claim 1, 2 or 3 wherein R⁵ is methyl, ethyl or allyl.

5. A compound as claimed in claim 1, 2, 3 or 4 wherein R⁶ is benzyl singly substituted by S(O)₂(C₁₋₄)alkyl or S(O)₂NR⁹R¹⁰; wherein R⁹ and R¹⁰ are, independently, hydrogen or C₁₋₄ alkyl, or together with a nitrogen or oxygen atom, join to form a 5- or 6-membered ring which is optionally substituted with C₁₋₄ alkyl, C(O)H or C(O)(C₁₋₄ alkyl).

6. A compound as claimed in claim 1, 2, 3, 4 or 5 wherein R⁷ is phenyl optionally substituted by halo, cyano, methyl, ethyl, methoxy, ethoxy, NH₂, NHCH₃, N(CH₃)₂, CF₃, CHF₂, CH₂F, CH₂CF₃ or OCF₃.

7. A compound as claimed in claim 1, 2, 3, 4, 5 or 6 wherein R⁸ is C₁₋₆ alkyl, C_{1≢}alkoxy, NR¹³R¹⁴, C₃₋₇ cycloalkyl (optionally substituted by C₁₋₄ alkyl) or heteroaryl; R¹³ is C₁₋₈ alkyl (optionally substituted by halogen, cyano, hydroxy, NH₂, N(C₁₋₄ alkyl)₂, C₁₋₄ alkoxy, C₁₋₄ thioalkyl, C₃₋₇ cycloalkyl, heterocyclyl, phenyl, heteroaryl, NHC(O)(C₁₋₄ alkyl) or C(O)NHOH), C₃₋₆ alkenyl, C₃₋₆ alkynyl, phenyl or heteroaryl; R¹⁴ is hydrogen, C₁₋₈ alkyl (optionally substituted by cyano or hydroxy) or C₃₋₆ alkenyl; or
R¹³ and R¹⁴ together with the nitrogen to which hey are attached form a oxiranyl, pyrrolidinyl, piperidinyl, morpholinyl, dihydropyrrolyl, tetrahydropyridinyl, piperazinyl, thiomorpholinyl, homopiperazinyl or homopiperidinyl ring all of which are optionally substituted by hydroxy, C₁₋₄ alkyl or C₁₋₄ hydroxyalkyl; wherein phenyl is optionally substituted by halogen, cyano, hydroxy or C₁₋₆ alkyl; and heteroaryl is optionally substituted by oxo, halogen, cyano, hydroxy or Clot alkyl.

8. A compound as claimed in claim 1, 2, 3, 4, 5, 6 or 7 wherein R⁹ is C₁₋₄ alkyl or C₃₋₄ alkenyl.

9. A processes for the preparation of a compound of formula (I) as claimed in claim 1 wherein R¹ is CHR⁷OC(O)R⁸ comprising reacting a compound of formula (II): 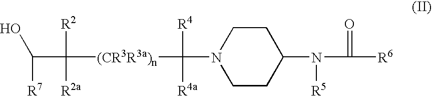
with a compound of formula R⁸C(O)Cl in the presence of a suitable base and in a suitable solvent.

10. A processes for the preparation of a compound of formula (I) as claimed in claim 1 wherein R¹ is CR⁷═NOR⁹ comprising reacting a compound of formula (III): 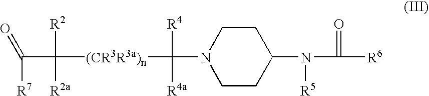
with a compound of formula R⁹ONH₂ in a suitable solvent.

11. A pharmaceutical composition which comprises a compound of the formula (I), or a pharmaceutically acceptable salt thereof or solvate thereof as claimed in claim 1 to 8, and a pharmaceutically acceptable adjuvant, diluent or carrier.

12. A compound of the formula (I), or a pharmaceutically acceptable salt thereof or solvate thereof as claimed in claim 1 to 8, for use in therapy.

13. A compound of formula (I), or a pharmaceutically acceptable salt thereof or solvate thereof as claimed in claim 1 to 8, in the manufacture of a medicament for use in therapy.

14. A compound of the formula (I): 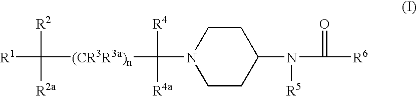
wherein: R¹ is a group selected from: 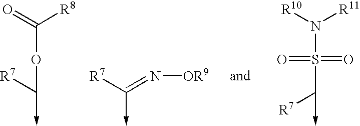
R², R^{2a}, R⁴ and R^{4s} are, independently, hydrogen or C₁₋₄ alkyl;
R³ and R^{3a} are, independently, hydrogen, C₁₋₄ alkyl or C₁₋₄ alkoxy;
n is 0 or 1;
R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;
R⁷ is phenyl, heteroaryl, phenyl(C₁₋₄ alkyl) or heteroaryl(C₁₋₄ alkyl);
R⁸ is C₁₋₈ alkyl, OR¹², NR¹³R¹⁴, phenyl, heteroaryl, phenyl(C₁₋₂)alkyl or heteroaryl(C₁₋₂)alkyl;
R⁹, R¹⁰ and R¹¹ are, independently, hydrogen, C₁₋₆ alkyl (optionally substituted by C₁₋₆ alkoxy, phenyl or heteroaryl), phenyl or heteroaryl; or R¹⁰ and R¹¹ may join to form a 5- or 6-membered ring which may additionally include an oxygen atom or a further nitrogen atom, said ring being optionally substituted with C₁₋₄ alkyl, C(O)H or C(O)(C₁₋₄ alkyl);
R¹² and R¹³ are C₁₋₈ alkyl (optionally substituted by halogen, OH, cyano, C₁₋₆ alkoxy, C₁₋₆ hydroxyalkoxy, C₁₋₆ alkylthio, C₃₋₆ cycloalkyl, NR¹⁵R¹⁶, C(O)NH(OH), NHC(O)(C₁₋₄ alkyl), heterocyclyl, phenyl or heteroaryl), C₃₋₆ alkenyl, C₃₋₆ alkynyl, C₃₋₆ cycloalkyl (optionally substituted by C₁₋₆ alkyl), phenyl, heteroaryl or heterocyclyl;
R¹⁴ is hydrogen or is independently selected from the list of options recited for R¹³;
or R¹³ and R¹⁴ join to form a 5, 6, 7 or 8-membered monocyclic or bicyclic ring system which is optionally unsaturated, optionally includes a further nitrogen atom or also includes an oxygen or sulphur atom, and which is optionally substituted by OH, C₁₋₆ alkyl or C₁₋₆ hydroxyalkyl;
R¹⁵ and R¹⁶ are, independently, hydrogen or C₁₋₆ alkyl;
wherein the phenyl, heteroaryl and heterocyclyl rings of any of the foregoing are independently optionally substituted by halo, cyano, nitro, oxo, hydroxy, C₁₋₄ alkyl, C₁₋₄ hydroxyalkyl, C₁₋₄ alkoxy, S(O)ₘC₁₋₄ alkyl, S(O)₂NR¹⁷R¹⁸, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R¹⁷ and R¹⁸ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, p and q are, independently, 0, 1 or 2;
or a pharmaceutically acceptable salt thereof or a solvate thereof; for use as a medicament, especially a medicament for the treatment of rheumatoid arthritis.

15. A method of treating a chemokine mediated disease state in a warm blooded animal suffering from, or at risk of, said disease, which comprises administering to an animal in need of such treatment a therapeutically effective amount of a compound of formula (I), or a pharmaceutically acceptable salt thereof or solvate thereof as claimed in claim 1 to 8, or as defined in claim 14.
